(19) United States
(12) Patent Application Publication
(10) Pub. No.: US 2014/0135561 A1
(43) Pub. Date: May 15, 2014

(54) PROTON BEAM GENERATION APPARATUS AND TREATMENT METHOD USING THE APPARATUS

(71) Applicant: Electronics and Telecommunications Research Institute, Daejeon (KR)

(72) Inventors: Dong Hoon SONG, Daejeon (KR); Moon Youn JUNG, Daejeon (KR)

(21) Appl. No.: 13/916,419

(22) Filed: Jun. 12, 2013

(30) Foreign Application Priority Data
Nov. 14, 2012 (KR) 10-2012-0128987

(51) Int. Cl. G21K 5/04; A61N 5/10

(52) U.S. Cl. CPC G21K 5/04; A61N 5/1077
USPC 600/1; 250/423 R

(57) ABSTRACT

Provided is a proton beam generation apparatus. The apparatus includes a laser system providing a laser pulse, a phase shift plate polarizing the laser pulse to be spirally radial to be transduced to be a spirally radial shape polarized laser pulse, and a target for generating a proton beam, generating a proton beam due to the spirally radial shape polarized laser pulse.

### CROSS-REFERENCE TO RELATED APPLICATIONS

This U.S. non-provisional patent application claims priority under 35 U.S.C. §119 of Korean Patent Application No. 10-2012-0128987, filed on Nov. 14, 2012, the entire contents of which are hereby incorporated by reference.

### BACKGROUND OF THE INVENTION

The present invention relates to a proton beam generation apparatus and a treatment method using the apparatus, and more particularly, to a proton beam generation apparatus using a laser beam and a treatment method using the apparatus.

There are X-ray treatment, electron beam treatment, and ion beam treatment as radiation treatments. Since being a cheapest method capable of being embodied by using a simplest apparatus, the X-ray treatment is most generally used now among radiation treatments. When injecting electrons into a tumor by accelerating electrons by using an accelerator, it is possible to treat the tumor, which was verified. However, miniaturization of electron accelerators was realized in 1980s in such a way that electron beam treatment has been regularly performed as one of radiation treatments. On the other hand, in the case of X-ray treatment or electron beam treatment, hydrogen bonds in a cancer cell are broken to destruct DNA but healthy cells existing in a progress pathway thereof are seriously damaged. To reduce radiation exposure to normal cells, intensity-modulated radiation therapy (IMRT) or tomo therapy, cyber knife, etc have been developed, which are incapable of perfectly prevent side effects as described above.

Ion beam treatment receives attention as treatment capable of reducing side effects in the X-ray treatment or electron beam treatment. To allow an ion beam to penetrate a material, as electrons, it is necessary to accelerate to allow the ion beam to have a high speed. When an ion beam penetrates a certain material, although a speed thereof is gradually decreased, there is present the greatest energy loss of ionizing radiation of the ion beam just before stop. This phenomenon is designated as Bragg peak named after William Henry Bragg who found the phenomenon in 1903. Accordingly, in the case of the ion beam treatment, when precisely controlling the speed of ions, it is possible to selectively, locally treat malignant tumors. When a tumor is located deeply inside the body, it is needed to accelerate protons or ions of very large amount of energy. As a method of accelerating protons or ions, there is a laser driven ion acceleration method. When irradiating a high-energy laser beam to a thin film, ions or protons inside the thin film escape from the thin film with acceleration energy due to due to a target normal sheath acceleration (TNSA) model or a radiation pressure acceleration (RPA) model. The ions escaping therefrom penetrates the body of a patient as the energy thereof and stop at a certain depth where the tumor is located and a large amount of free oxygen radicals are generated in such a way that malignant cells necrotize, which becomes a theory of general ion beam treatments.

In the case of ion beam treatment using the laser driven acceleration method, there are two necessary features of ions. To inject ions deeply into the body, it is necessary that ions are in a high-energy state and most ions have the same energy. Protons penetrate the body by 20 cm at 250 MeV of energy. In the case of treating an ocular cancer, ions having high energy of 70 MeV are needed. Also, to treat cancers deep inside the body, ions having high energy more than 200 MeV are needed.

Also, it is necessary that energies of most protons or ions driven by femtosecond-laser are the same. When the energies are not the same, since the ions are not integrated on a location of a tumor, normal tissues may be exposed to the ions.

### SUMMARY OF THE INVENTION

The present invention provides a proton beam generation apparatus capable of obtaining a proton beam having uniform energy and simultaneously with improving productivity.

The present invention also provides a treatment method using a proton beam generation apparatus capable of obtaining a proton beam having uniform energy and simultaneously with improving productivity.

Embodiments of the present invention provide proton beam generation apparatuses including a laser system providing a laser pulse, a phase shift plate polarizing the laser pulse to be spirally radial to be transduced to be a spirally radial shape polarized laser pulse, and a target for generating a proton beam, generating the proton beam due to the spirally radial shape polarized laser pulse.

In some embodiments, the phase shift plate may include a plurality of sectors obtained by dividing a circular plate.

In other embodiments, the sectors may be first to nth half phase plates.

In still other embodiments, the first to nth half phase plates may be zeroth-order half phase plates.

In even other embodiments, the half phase plates may include a nonlinear material having double refraction with respect to the laser pulse.

In yet other embodiments, the nonlinear material may include one of crystal quartz or polymers.

In further embodiments, the first to nth half phase plates may have optical axes gradually rotating according to a rotational direction of the circular plate.

In still further embodiments, the respective second to nth phase plates may have optical axes gradually integer times of a certain angle with respect to an optical axis of the first half phase plate.

In even further embodiments, the laser system may include chirped pulse amplification (CPA) module.

In yet further embodiments, the CPA module may include a source generating the laser pulse and an amplifier amplifying the strength of the laser pulse.

In much further embodiments, the source may include a titanium-sapphire crystal gain medium.

In still much further embodiments, the amplifier may include the same gain medium as the source.

In even much further embodiments, the CPA module may further include a pulse stretcher increasing a pulse width of the laser pulse between the source and the amplifier, and a compressor decreasing the pulse width of the laser pulse between the amplifier and the phase shift plate.

In yet much further embodiments, the pulse stretcher may include Öffner-triplet type reflecting optical system.

In other embodiments, the Öffner-triplet type reflecting optical system may include a pair of diffraction gratings diffracting the laser pulse, a plurality of convex lenses disposed between the first diffraction gratings, a first input/output mirror inputting and outputting the laser pulse to one of the first diffraction gratings, and a first concave mirror reflecting the laser pulse to another of the first diffraction gratings.

In other embodiments, the compressor may include a pair of second diffraction gratings diffracting the laser pulse, a second input/output mirror inputting and outputting the laser pulse diffracted from one of the second diffraction gratings, and a second concave mirror reflecting the laser pulse to another of the second diffraction gratings.

In other embodiments, the CPA module may further include one of a plurality of mirrors and a plurality of half mirrors disposed among the source, the pulse stretcher, the amplifier, the compressor, and the phase shift plate and transmitting the laser pulse.

In other embodiments, the target for generating the proton beam may include a material including one of hydrogen and carbon.

In other embodiments of the present invention, treatment methods include projecting a proton beam generated by the proton beam generation apparatus toward a tumor portion of a patient.

In some embodiments, the target for generating the proton beam may include a material including one of hydrogen and carbon.

### BRIEF DESCRIPTION OF THE DRAWINGS

The accompanying drawings are included to provide a further understanding of the present invention, and are incorporated in and constitute a part of this specification. The drawings illustrate exemplary embodiments of the present invention and, together with the description, serve to explain principles of the present invention. In the drawings:

FIG. 1 is a schematic configuration view illustrating a proton beam generation apparatus according to an embodiment of the present invention;

FIG. 2 is a three-dimensional view illustrating a laser pulse transduced by the proton beam generation apparatus of FIG. 1;

FIG. 3 is a photograph illustrating the laser pulse transduced by the proton beam generation apparatus of FIG. 1;

FIG. 4 is a configuration view illustrating the proton beam generation apparatus of FIG. 1;

FIGS. 5 and 6 are top views illustrating examples of the proton beam generation apparatus of FIG. 1; and

FIG. 7 is a schematic configuration view illustrating a method of performing treatment by using the proton beam generation apparatus of FIG. 1.

### DETAILED DESCRIPTION OF PREFERRED EMBODIMENTS

Hereinafter, the embodiments of the present invention will be described in detail with reference to the attached drawings. Advantages, features, and a method of achieving the same will be specified with reference to embodiments that will be described below in detail with reference to the attached drawings.

However, the present invention is not limited to the embodiments described below and may be embodied in other forms. The embodiments that will be described hereafter are provided to allow the disclosure to be thoroughgoing and perfect and to allow a person skilled in the art to fully understand the scope of the present invention. Through the entire specification, like reference numerals designate like elements.

Terms are used in the specification to describe the embodiments but not to limit the scope of the present invention. In the specification, a singular form includes a plural form if there is no particular mention. “Comprises” and/or “comprising” used in the specification do or does not the existence or addition of one or more other elements, steps, operations, and/or devices in addition to an element, a step, an operation, and/or a device, which are mentioned. Also, as just exemplary embodiments, reference numerals shown according to an order of description are not limited thereto. Additionally, in the specification, when it is mentioned that a film is on another film or a substrate, the film may be formed directly on the other film or the substrate or still another film may be disposed therebetween.

Also, the embodiments described in the specification will be explained with reference to cross-sectional views and/or top views. In the drawings, thicknesses of a film and an area may be exaggerated to effectively explain technical contents. Accordingly, shapes shown in the drawings may be changed by manufacturing technology and/or tolerable errors. Accordingly, the embodiments of the present invention are not limited to certain shapes but include variances in shapes formed according to a manufacturing process. For example, an etching area shown in a right-angled shape may be rounded or may be a shape having a certain curvature. Accordingly, areas shown in the drawings have schematic properties and shapes of areas shown in the drawings are just to illustrate certain shapes of elements but not to limit the scope of the present invention.

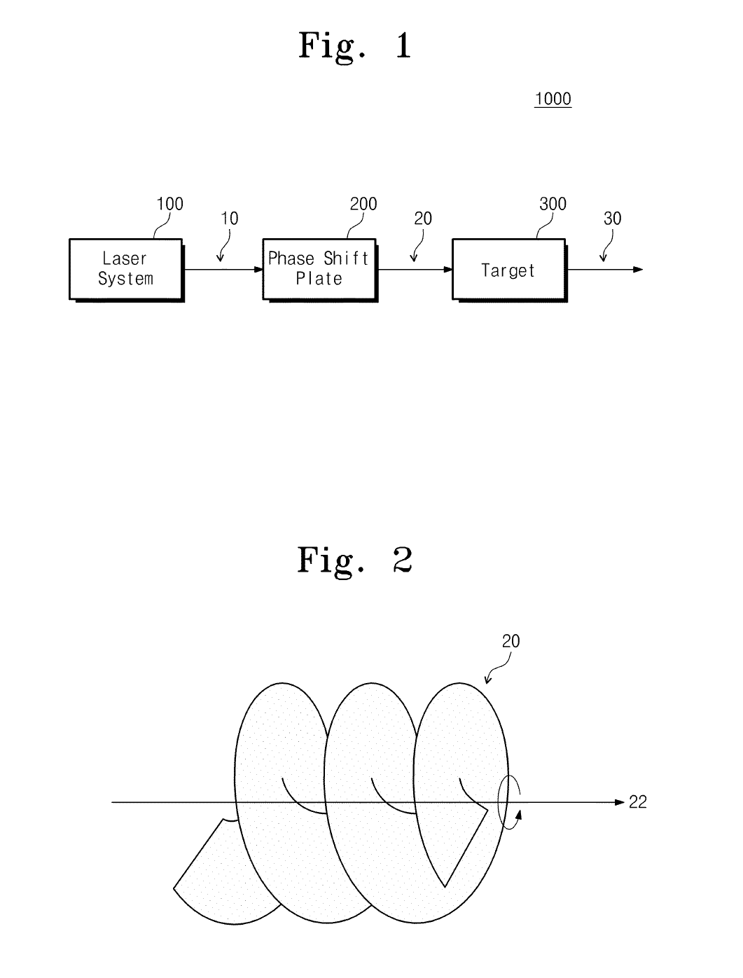

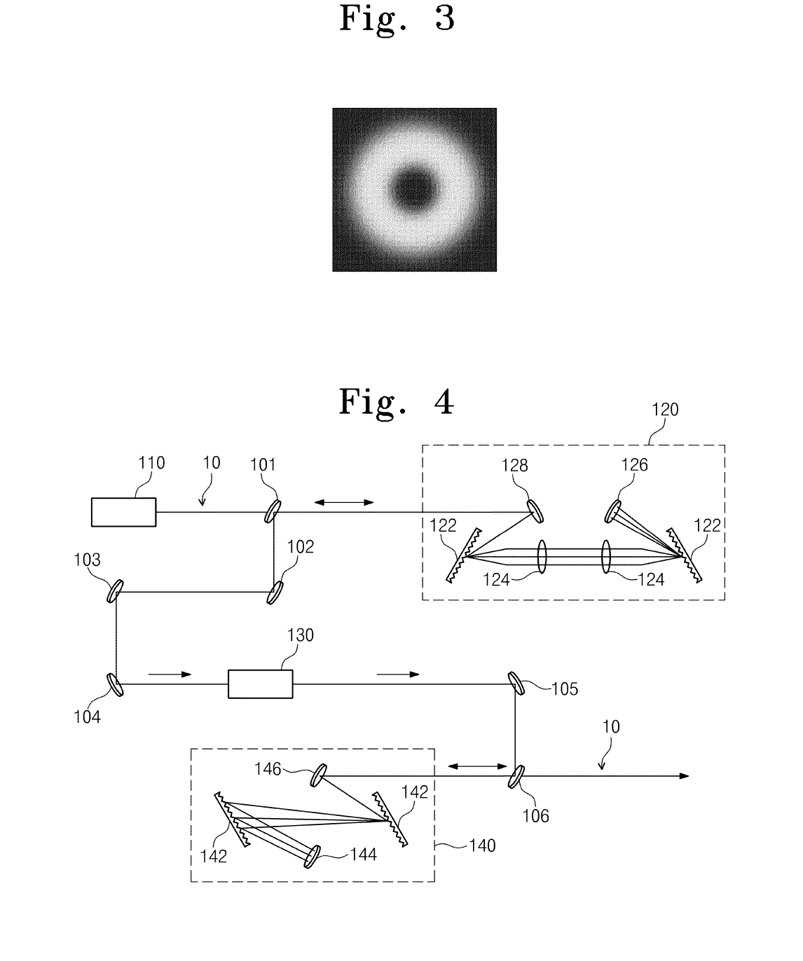

FIG. 1 is a schematic configuration view illustrating a proton beam generation apparatus according to an embodiment of the present invention, FIG. 2 is a three-dimensional view illustrating a laser pulse transduced by an element of the proton beam generation apparatus, and FIG. 3 is a photograph illustrating the laser pulse transduced by the element of the proton beam generation apparatus.

Referring to FIGS. 1 to 3, a proton beam generation apparatus 1000 may include a laser system 100, a phase shift plate 200, and a target 300 for generating a proton beam.

The laser system 100 may include chirped pulse amplification (CPA) module (refer to FIG. 4) generating a lineally polarized laser pulse 10.

The phase shift plate 200 may convert the lineally polarized laser pulse 10 generated by the laser system 100 into a radially polarized laser pulse 20 having a spiral-donut shape. The radially polarized laser pulse 20 may be incident, in the shape of a spiral donut with respect to a progress direction 22, to the target 300 for generating the proton beam. FIG. 3 is the photograph taking an image of the radially polarized laser pulse 20 in the progress direction 22, which shows that the radially polarized laser pulse 20 has a donut shape.

The target 300 is a source of a proton beam 30 and may include a material that includes hydrogen or carbon. A material including hydrogen may be silicon nitride, silicon oxide, or metal. A material including carbon may include graphene. The radially polarized laser pulse 20 may generate a larger amount of the proton beam 30 having uniform energy, relative to the linearly polarized laser pulse 10.

Accordingly, the proton beam generation apparatus 1000 may have improved productivity.

FIG. 4 is a configuration view illustrating the proton beam generation apparatus.

Referring to FIGS. 1 to 4, the laser system 100 of the proton beam generation apparatus 1000 may include a source 110, a pulse stretcher 120, an amplifier 130, and a compressor 140.

The source 110 may generate the linearly polarized laser pulse 10 having an ultrahigh-frequency wavelength. For example, the source 110 may include a titanium-sapphire crystal medium generating a laser beam having P-polarized light pulse having a wavelength within a range from about 650 nm to about 1,100 nm but is not limited thereto. The source 110 may be an ultrahigh-frequency laser including another gain medium. The P-polarized pulse is the linearly polarized laser pulse 10.

A first half mirror 101 may be disposed between the source 110 and the pulse stretcher 120. The first half mirror 101 may penetrate the linearly polarized laser pulse 10 progressing from the source 110 to the pulse stretcher 120.

The pulse stretcher 120 may increase a pulse width of the linearly polarized laser pulse 10. The pulse stretcher 120 may increase a femtosecond pulse width to a picosecond or nanosecond pulse width by using a positive high order dispersion value. The pulse stretcher 120 may include an Öffner-triplet type reflecting optical system. The Öffner-triplet type reflecting optical system may include a pair of first diffraction gratings 122, convex lenses 124 between the first diffraction gratings, a concave mirror 126, and a first input/output mirror 128.

The first diffraction gratings 122 may determine an increased wavelength of the linearly polarized laser pulse 10. The first diffraction gratings 122 may face each other to be nonparallel or parallel to each other. The convex lenses 124 may focus the linearly polarized laser pulses 10 on the first diffraction gratings 122, respectively. It may be known that the linearly polarized laser pulses 10 between the convex lenses 124 may be parallel laser beams. The first diffraction gratings 122 may diffract the linearly polarized laser pulses 10. The concave lens 126 may reflect the linearly polarized laser pulses 10 diffracted from one of on the first diffraction gratings 122. The concave lens 126 may reflect the linearly polarized laser pulses 10 having the form of parallel laser beams to the first diffraction gratings 122. In this case, the concave lens 126 may have a radius curvature two times of that of the convex lenses 124. The first input/output mirror 128 may reflect the linearly polarized laser pulse 10 from the source 110 to another of the first diffraction gratings 122, and simultaneously, may reflect the linearly polarized laser pulse 10 in a direction from the first diffraction gratings 122 to the source 110. Since the linearly polarized laser pulse 10 that is reflected again has a different height, there is no interference.

In this case, the pulse stretcher 120 may generate the linearly polarized laser pulse 10 having a picosecond or nanosecond pulse width increased according to a positive group delay dispersion value. The group delay dispersion value may be determined by using diffraction angles and grating constants of the first diffraction gratings 122, an incident angle of the first input/output mirror 128, and a function with respect to a distance between the first diffraction gratings 122 and the concave mirror 126. Accordingly, the pulse stretcher 120 may provide the linearly polarized laser pulse 10 whose pulse width is more stretched than the source 110 to the amplifier 130 through reflections by the first half mirror 101, a second mirror 102, a third mirror 103, and a fourth mirror 104.

The pulse stretcher 120 may increase power of the linearly polarized laser pulse 10. The amplifier 130 may include the same gain medium as the source 110. The gain medium may generate the linearly polarized laser pulse 10 with high power by using the linearly polarized laser pulse 10 provided from the pulse stretcher 120 as a seed light. The source 110 may provide the linearly polarized laser pulse 10 having the same wavelength as the source 110. A fifth mirror 105 and a second half mirror 106 may reflect the linearly polarized laser pulse 10 from the amplifier 130 to the compressor 140.

The pulse stretcher 140 may decrease the pulse width of the linearly polarized laser pulse 10 by using a negative dispersion value. The compressor 140 may output the linearly polarized laser pulse 10 as femtoseconds. For example, the compressor 140 may include a pair of second diffraction gratings 142, a second concave mirror 144, and a second input/output mirror 146. The second diffraction gratings 142 may have a small interval therebetween than the first diffraction gratings 122. The second input/output mirror 146 may allow the linearly polarized laser pulse 10 to be incident to one of the second diffraction gratings 142. The second diffraction gratings 142 and the second concave mirror 144 may decrease the pulse width of the linearly polarized laser pulse 10. The second diffraction gratings 142 may diffract the linearly polarized laser pulse 10 to be incident to the second concave mirror 144. The second concave mirror 144 may reflect the diffracted linearly polarized laser pulse 10 to the second diffraction gratings 142.

As described above, the wavelength of the linearly polarized laser pulse 10 may be shortened by the negative dispersion value. The dispersion value may be determined by using grating constants and diffraction angles of the second diffraction gratings 142, an incident angle of the first input/output mirror 146, and a distance between the second diffraction gratings 142. The second half mirror 106 may provide the linearly polarized laser pulse 10 outputted from the second input/output mirror 146 of the compressor 140 to the phase shift plate 200.

Accordingly, the laser system 100 of the proton beam generation apparatus 1000 may provide the linearly polarized laser pulse 10 with high power to the phase shift plate 200.

The phase shift plate 200 of the proton beam generation apparatus 1000 may transduce the high-power linearly polarized laser pulse 10 into the spirally radial shape polarized laser pulse 20 having orbital angular momentum. In this case, the orbital angular momentum is a physical value designating quantized linearly polarized laser pulse 10 and has no unit. The spirally radial shape polarized laser pulse 20 may have orbital angular momentum integer times of 2π radian of 360° or integer times of π radian.

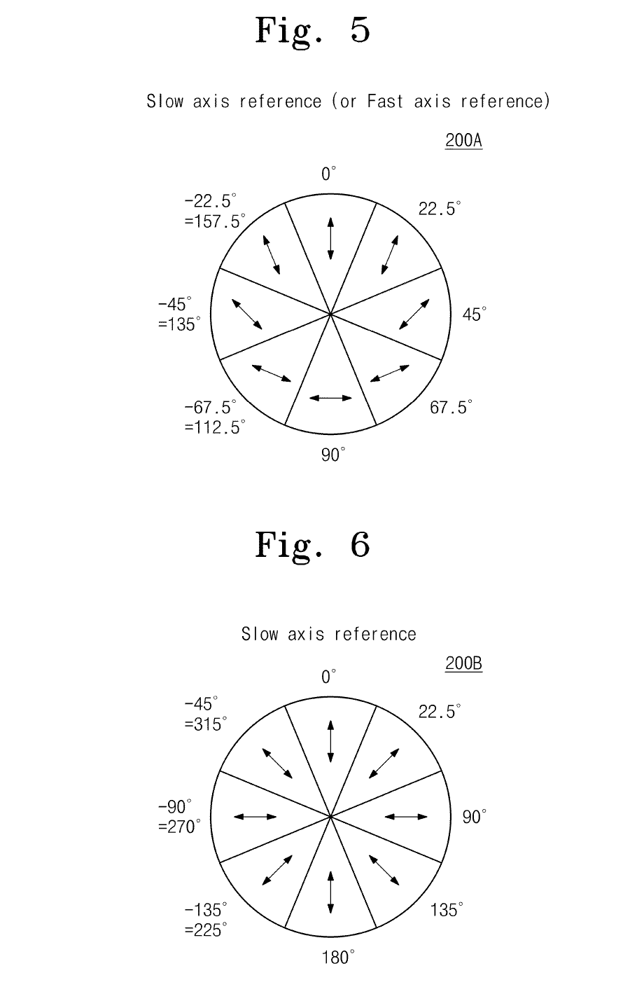

For example, the phase shift plate 200 may be configured as shown in FIG. 5 or 6, depending on the quantity of the orbital angular momentum of the spirally radial shape polarized laser pulse 20.

Referring to FIG. 5, a phase shift plate 200A may include a plurality of sectors dividing a circular plate in a direction of azimuth. The sectors may include a nonlinear material having double refraction. The nonlinear material may include crystal quartz or polymers. The sectors may include first to eighth half phase plates. The first to eighth half phase plates may be zeroth-order half phase plates. The first to eighth half phase plates may be obtained by dividing 360° circular plate by 45°. The second to eighth half phase plates may have optical axes gradually increasing with a certain angle such as 22.5° in response to an optical axis of the first half phase plate, 0° with a slow axis or a fast axis as a reference, respectively. A linearly polarized laser pulse (refer to the reference numeral 10 in FIG. 1) may be transduced into a spirally radial shape polarized laser pulse (refer to the reference numeral 20 in FIG. 2) having the donut shape. Orbital angular momentum L of the spirally radial shape polarized laser pulse may be 1. The phase shift plate 200A includes first to nth half phase plates, and the spirally radial shape polarized laser pulses having higher efficiency may be provided as the number of the first to nth half phase plates more increases.

Referring to FIG. 6, a phase shift plate 200B may include a plurality of sectors dividing a circular plate in a direction of azimuth. The sectors may include a nonlinear material having double refraction. The nonlinear material may include crystal quartz or polymers. The sectors may include first to eighth half phase plates. The first to eighth half phase plates may be zeroth-order half phase plates. The first to eighth half phase plates may be obtained by dividing 360° circular plate by 45°. The second to eighth half phase plates may have optical axes gradually increasing with a certain angle such as 22.5° in response to an optical axis of the first half phase plate, 0° with a slow axis or a fast axis as a reference, respectively. A linearly polarized laser pulse (refer to the reference numeral 10 in FIG. 1) may be transduced into a spirally radial shape polarized laser pulse (refer to the reference numeral 20 in FIG. 2) having the donut shape. Orbital angular momentum L of the spirally radial shape polarized laser pulse may be 2. The phase shift plate 200B includes first to nth half phase plates, and the spirally radial shape polarized laser pulses having higher efficiency may be provided as the number of the first to nth half phase plates more increases.

Accordingly, the phase shift plates 200A and 200B may transduce the linearly polarized laser pulse into the spirally radial shape polarized laser pulse having various orbital angular momentums. The radially polarized laser pulse may generate a proton beam (refer to the reference numeral 30 in FIG. 1) having a function according to the quantity of orbital angular momentum from a target for generating the proton beam (refer to the reference numeral 300 in FIG. 1). Accordingly, the proton beam generation apparatus 1000 may have improved productivity.

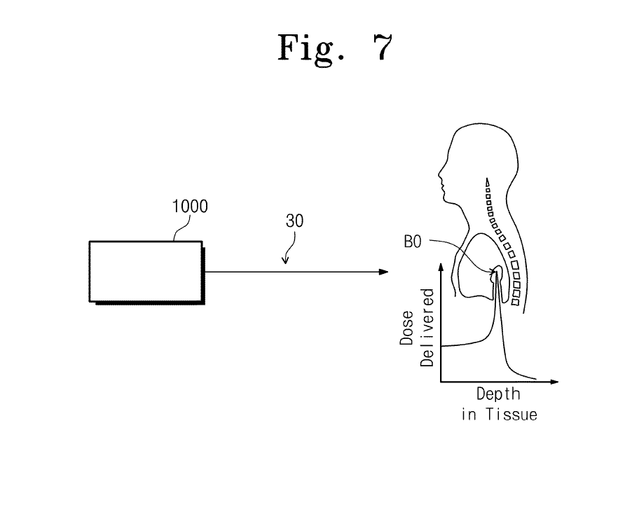

FIG. 7 is a schematic configuration view illustrating a method of performing treatment by using the proton beam generation apparatus.

Referring to FIG. 7, a proton beam 30 may be emitted from a target (refer to the reference numeral 300 in FIG. 1) due to a spirally radial shape polarized laser pulse (refer to the reference numeral 20 in FIG. 1) generated by a phase shift plate (refer to the reference numeral 200 in FIG. 1).

When the spirally radial shape polarized laser pulse is provided to the target, atoms contained in the target go through an ionization process in such a way that protons are formed as the proton beam 30 and may be projected to a tumor portion B0 inside a human body. That is, the proton beam 30 generated from the target stops at the tumor portion B0 inside the body of a patient and may collide with the tumor portion B0.

The proton beam 30 may be set to a location of the tumor portion B0 obtained from an imaging diagnosis apparatus such as magnetic resonance imaging (MRI) apparatus, a computer tomography (CT) apparatus, a positron emission tomography (PET) apparatus, and an ultrasonic wave apparatus, which is used to diagnose the tumor portion B0 of the patient, and may be projected thereto.

In a therapeutic theory using a proton beam generation apparatus, a linearly polarized laser pulse (refer to the reference numeral 10 in FIG. 10) provided from a laser system (refer to the reference numeral 100 in FIG. 1) may be transduced into a spirally radial shape polarized laser pulse by a phase shift plate, the proton beam 30 may be generated from a target for generating a proton beam due to the spirally radial shape polarized laser pulse and may be projected toward the inside of a body of a patient, and the proton beam 30 projected to the inside of the body of the patient, as shown in the drawing, may stop at the tumor portion B0 and may collide with the tumor portion B0, thereby generating active oxygen radicals to disturb tumor cells.

That is, the proton beam 30 may collide with the tumor portion B0 and may generate active oxygen radicals to disturb tumor cells, thereby hindering growth of tumor cells or necrotizing tumor cells. Disturbing tumor cells of the tumor portion B0 by the proton beam 30 means disturbing the double helix of DNA of a tumor cell or disturbing a metabolic process inside the nucleus of the tumor cell.

In processes of generating and projecting the proton beam 30, when a spirally radial shape polarized laser pulse is incident to a target for generating a proton beam, atoms of hydrogen or ions of carbon included in the target are changed into a plasma state of being separated into positive ions (not shown) and negative ions (not shown) by energy of the spirally radial shape polarized laser pulse, in which the negative ions are separated from the target further than the positive ions, thereby generating an electric field due to a capacitor effect between the positive ions and the negative ions, and the positive ions are accelerated toward the negative ions by the electric field in such a way that the proton beam 30 formed of the positive ions may be accelerated while having full energy to be projected from the outside of the patient to the tumor portion B0 inside the body of the patient.

The accelerated proton beam 30 may collide with the tumor portion B0 inside the body of the patient and may generate active oxygen radicals to disturb tumor cells of the tumor portion B0, thereby hindering growth of the tumor cells or necrotizing the tumor cells. According thereto, an effect of treating the tumor portion B0 inside the body of the patient may be shown.

The proton beam generation apparatus 1000 includes a phase shift plate transducing a linearly polarized laser pulse into a spirally radial shape polarized laser pulse between a laser system and a target for generating a proton beam, thereby generating a large amount of proton beams having uniformly dispersed energy from the target by generating a laser pulse having a spiral phase in the shape of a donut. Accordingly, it is possible to provide the proton beam generation apparatus capable of improving productivity.

Also, according to the present embodiment, since the treatment method uses the proton beam generation apparatus capable of generating a large amount of proton beams having uniformly dispersed energy, a proton beam having the uniformly dispersed energy may be projected to a tumor portion of a patient. According thereto, it is possible to provide a treatment method capable of efficiently treating a tumor of a patient.

Also, since the treatment method using the proton beam generation apparatus generates a large amount of proton beams having uniformly dispersed energy and uses a target for generating ions, having an ultra thin film having a bubble shape with high energy, thereby projecting the proton beam having uniformly dispersed energy toward a tumor portion of a patient. According thereto, it is possible to provide a treatment method capable of efficiently treating a tumor of the patient.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A proton beam generation apparatus comprising:
a laser system providing a laser pulse;
a phase shift plate polarizing the laser pulse to be spirally radial to be transduced to be a spirally radial shape polarized laser pulse; and
a target for generating a proton beam, generating the proton beam due to the spirally radial shape polarized laser pulse.

2. The apparatus of claim 1, wherein the phase shift plate comprises a plurality of sectors obtained by dividing a circular plate.

3. The apparatus of claim 2, wherein the sectors are first to nth half phase plates.

4. The apparatus of claim 3, wherein the first to nth half phase plates are zeroth-order half phase plates.

5. The apparatus of claim 3, wherein the half phase plates comprise a nonlinear material having double refraction with respect to the laser pulse.

6. The apparatus of claim 5, wherein the nonlinear material comprises one of crystal quartz or polymers.

7. The apparatus of claim 3, wherein the first to nth half phase plates have optical axes gradually rotating according to a rotational direction of the circular plate.

8. The apparatus of claim 3, wherein the respective second to nth phase plates have optical axes gradually integer times of a certain angle with respect to an optical axis of the first half phase plate.

9. The apparatus of claim 1, wherein the laser system comprises chirped pulse amplification (CPA) module.

10. The apparatus of claim 7, wherein the CPA module comprises:
a source generating the laser pulse; and
an amplifier amplifying a strength of the laser pulse.

11. The apparatus of claim 10, wherein the source comprises a titanium-sapphire crystal gain medium.

12. The apparatus of claim 11, wherein the amplifier comprises the same gain medium as the source.

13. The apparatus of claim 10, wherein the CPA module further comprises:
a pulse stretcher increasing a pulse width of the laser pulse between the source and the amplifier; and
a compressor decreasing the pulse width of the laser pulse between the amplifier and the phase shift plate.

14. The apparatus of claim 13, wherein the pulse stretcher comprises Öffner-triplet type reflecting optical system.

15. The apparatus of claim 14, wherein the Öffner-triplet type reflecting optical system comprises:
a pair of diffraction gratings diffracting the laser pulse;
a plurality of convex lenses disposed between the first diffraction gratings;
a first input/output mirror inputting and outputting the laser pulse to one of the first diffraction gratings; and
a first concave mirror reflecting the laser pulse to another of the first diffraction gratings.

16. The apparatus of claim 13, wherein the compressor comprises:
a pair of second diffraction gratings diffracting the laser pulse;
a second input/output mirror inputting and outputting the laser pulse diffracted from one of the second diffraction gratings; and
a second concave mirror reflecting the laser pulse to another of the second diffraction.

17. The apparatus of claim 13, wherein the CPA module further comprises one of a plurality of mirrors and a plurality of half mirrors disposed among the source, the pulse stretcher, the amplifier, the compressor, and the phase shift plate and transmitting the laser pulse.

18. The apparatus of claim 1, wherein the target for generating the proton beam comprises a material comprising one of hydrogen and carbon.

19. A treatment method comprising projecting a proton beam generated by a proton beam generation apparatus toward a tumor portion of a patient, the proton beam apparatus comprising:
a laser system providing a laser pulse;
a phase shift plate polarizing the laser pulse to be spirally radial to be transduced to be a spirally radial shape polarized laser pulse; and
a target for generating the proton beam, generating the proton beam due to the spirally radial shape polarized laser pulse.

20. The treatment method of claim 19, wherein the target for generating the proton beam comprises a material comprising one of hydrogen and carbon.
